(19) United States
(12) Patent Application Publication
(10) Pub. No.: US 2009/0226544 A1
(43) Pub. Date: Sep. 10, 2009

(54) Pharmaceutical Compound For Treating Inflammation, Pain, Arthritis And Spinitis, And Proliferating Osteoblastic Cell And Method For Producing Thereof

(76) Inventors: Jun- Sik Shin, Seoul (KR); Sun-Mee Lee, Seoul (KR)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 11/922,767

(22) PCT Filed: Jun. 27, 2006
(86) PCT No.: PCT/KR2006/002500

§ 371 Date: Dec. 19, 2007

(30) Foreign Application Priority Data
Jun. 28, 2005 (KR) 10-2005-0056167

(51) Int. Cl. A61K 36/00

(52) U.S. Cl. 424/725

(57) ABSTRACT

The present invention relates to a pharmaceutical composition having efficacy in the treatment of inflammation, pain, arthritis and spinitis, and the proliferation and activity of ost eoblastic cells, and a preparation method thereof. The present invention provides a pharmaceutical composition comprising pharmaceutically effective components extracted from Cibotii Rhizoma, Ledebouriellae Radix, Achyranthes bidentatae Radix, Acanthopanacis Cortex, Eucommiae Cortex, and Glycine Semen nigra, and a preparation method thereof. The pharmaceutically effective components may be extracted using hot water, an organic solvent, or a mixed solvent thereof. Alternatively, the pharmaceutically effective components may be prepared by a UF membrane to have a molecular weight of not greater than 10,000.

### TECHNICAL FIELD

The present invention relates to a pharmaceutical composition comprising pharmaceutically effective components extracted from Cibotii rhizoma, Ledebouriellae Radix, Achyranthes bidentatae Radix, Acanthopanacis Cortex, Eucommiae Cortex and Glycine Semen nigra, and a preparation method thereof.

The pharmaceutical composition according to the present invention is effective in the treatment of inflammation, pain, arthritis and spinitis, and the proliferation and ALP activity of osteoblastic cells.

### BACKGROUND ART

Non-steroidal anti-inflammatory drugs (NSAIDs) such as phenybutazone, diclofenac, or aceclofenac, are well-known drugs for the treatment of pain and inflammation. One of the major drawbacks of NSAIDs is that they may produce severe adverse side effects associated with long-term administration.

In view of the above-mentioned problems, to replace conventional NSAIDs with novel ones, active research is ongoing on novel pharmaceutical formulations that are effective in the treatment of inflammation and alleviation of pain, which by isolating pharmacologically active agents from raw substance of oriental medicine.

Korean Patent Publication Gazette No. 10-180567 discloses a pharmaceutical composition having excellent effects in treatment or prevention of inflammation and pain, chronic rheumatoid arthritis and ruptured disc, promotion of blood circulation, and so on.

After a long period of research, the present inventor successfully isolated from a Chinese medicinal plant Cibotii Rhizoma a novel compound 2-O-(9z,12z-octadecadienyl)-3-O-[α-galactopyranosyl-(1″-6′)-O-β-D-galactopyranosyl] glycerol, which is named shinbarometzin. Also, the present inventor developed pharmaceutical preparations having therapeutic efficacy on arthritis, osteoporosis and ruptured disc from Cibotii Rhizoma and other oriental medicinal compositions, which are disclosed in Korean Patent Registration Nos. 10-396857 and 10-415826, and U.S. Pat. No. 6,531,582.

The present inventor has endeavored to study novel oriental medicinal substance (natural form) which can replace NSAIDs and finally developed a pharmaceutical-composition, which is effective in treating inflammation, pain, osteoporosis, arthritis and ruptured disc using oriental medicinal substance such as Cibotii Rhizoma, Ledebouriellae Radix, Achyranthes bidentatae Radix, Acanthopanacis Cortex, Eucommiae Cortex, or Glycine Semen nigra, and proliferating or activating osteoblastic cells, completing the present invention.

Cibotii rhizoma is Cibotium barometz J. Smith of which the rhizoma is used for the medicinal purpose and which inhabits a tropical region and is a medicinal plant belonging to Dicksoniaceae [Guidelines listed in the Korean Herbal Pharmacopoeia, compiled by H. J. Ji and S. I. Lee, Korea Medical Index Corp., p. 79 (1988)]. According to the reference relating to Chinese medicines, Cibotii rhizoma has an effect of alleviating the joint pain and strengthening muscles and skeletons and has been disclosed that it has been used as a popular remedy. It has been known that Cibotii rhizoma contains onitin, onitin 4-O-β-D-allopyranoside, onitin 4-O-β-di-glucopyranoside and pterosin R (4-deoxy, 4-chloro onitin) and further, onitin has an activity for relaxing smooth muscles [Murakami, Takao; Satake, Toshiko; Ninomiya, Katsumi; lida, Hideki; Yamauchi, Kazuhiko; Tanaka, Nobotoshi; Saiki, Yasuhisa; Chen, Chiu-Ming, Pterosin-derivate aus der Famile Pteridaceae. Phytochemistry, 19, 1743 1980]. Yang, Meei-Shieu, Studies on the Twian fork medicine VI. Studies on onitin. Planta Medica, p. 25 (1986)].

Ledebouriellae Radix is a perennial plant, which belongs to the order of dropwort, the family of dropwort, and inhabits a grassland or a stony mountainous district, including Korea, China (Northeast area and North China), Mongolia, Siberia, etc. The root of Ledebouriellae Radix is, commonly referred to as Seseleos Radix in the oriental medicine, has been used as a hidrotic, antipyretic, analgesic, spasmolytic or diuretic medicament for preventing or treating cold, headache, arthritis, carpopedal spasm, tetanus, and so on.

Achyranthes bidentatae Radix is a perennial plant of Achyranthes japonica, Achyranthes bidentata, Cyathula officinalis and Cyathula capitata that belong to Amaranthaceae and contains large quantities of saponin and calcium as main ingredients.

Owing to its analgesic effect, it has been used for treating knee diseases such as arthritis or rheumatoid arthritis and inflammation due to bruise.

Acanthopanacis Cortex is a root bark of Acanthopanax gracilistylus W. W. SMITH and the same genus plants, which is a deciduous tree belonging to the family of Araliaceae, and has been used as a tonic medicament for a long time in the oriental medicine. It has been known that Acanthopanacis Cortex has efficacy of augmenting muscles and bones, preventing or treating inflammation and pain, and promoting blood circulation.

Eucommiae Cortex is a deciduous tree which belongs to Eucommiaceae, originated in China and is cultivated in Korea and Japan. The bark of the tree, commonly referred to as Cortex eucommiae, has been used for natural medicine since ancient times.

Glycine Semen nigra has been known that it has a detoxication function and toxicosis from poisoning can be treated by boiled Glycine Semen nigra.

### DISCLOSURE OF INVENTION

### Technical Problem

To solve the above problems, it is an objective of the present invention to provide a pharmaceutical composition, and a preparation method thereof, the pharmaceutical composition being extracted from naturally occurring raw medicinal substance, which is pharmacologically stable, and having effects in treating inflammation, pain, arthritis and spinitis, and proliferating or activating osteoblastic cells, unlike the conventional NSAIDs which may produce unwanted side effects when long-term administration is involved.

### Technical Solution

As used herein, the term “pharmaceutical composition” may refer to a pharmaceutical solid, a concentration, powder, a tablet, a capsule, or the like. As used herein, the term “pharmaceutically effective component” may refer to substance which effectively suppresses, relieves or treats medical symptoms. Here, the medical symptoms may comprise inflammation, pain, arthritis, spinitis, and loss of osteoblastic cells.

As used herein, the term “raw substance” may refer to a pharmaceutical material extracted from Cibotii Rhizoma, Ledebouriellae Radix, Achyranthes bidentatae Radix, Acanthopanacis Cortex, Eucommiae Cortex, or Glycine Semen nigra.

As used herein, the term “raw substance mixture” may refer to a mixture comprising at least one selected from the group consisting of Cibotii rhizoma, Ledebouriellae Radix, Achyranthes bidentatae Radix, Acanthopanacis Cortex, Eucommiae Cortex, and Glycine Semen nigra.

As used herein, the term “hot water” may refer to water or vapor of 100° C. or higher and the term “filtration using hot water” may refer to filtration performed by reflux and cool raw substance or raw substance mixture.

As used herein, the term “organic solvent” may refer to lower alcohol such as methanol, ethanol or buthanol, and “mixed solvent thereof” may refer to a mixed solvent of water and an organic solvent.

As used herein, the term “Ultrafiltration(UF)” may refer to isolation of materials having a molecular weight of not greater than 10,000.

The present invention provides a pharmaceutical composition comprising a pharmaceutically effective component extracted from Cibotii Rhizoma, Ledebouriellae Radix, Achyranthes bidentatae Radix, Acanthopanacis Cortex, Eucommiae Cortex, and Glycine Semen nigra. The pharmaceutical composition is effective in treating inflammation, pain, arthritis and spinitis, and proliferating or activating osteoblastic cells.

In the raw substance mixture, Cibotii Rhizoma, Ledebouriellae Radix, Achyranthes bidentatae Radix, Acanthopanacis Cortex, Eucommiae Cortex, and Glycine Semen nigra are preferably present in the ratio by weight of 1:0.5˜3:0.5˜3:0.5˜3:0.1˜2:0.5˜2, more preferably, in the ratio by weight of 1:1˜2:12:12:0.3˜1:0.7˜1.5. Before extracting the pharmaceutically effective components, the raw substance mixture present in the ratio by weight is pulverized to be powdered.

Hot water, an organic solvent, or a mixed solvent thereof can be used as a solvent for extracting the pharmaceutically effective component from the raw substance mixture. Extracting using hot water may be performed in the following manner. That is, about 8 to 50-fold distilled water based on a total weight of the raw substance mixture is refluxed at about 100° C. for about 3 hours and cooled for extraction. Lower alcohol such as methanol, ethanol or buthanol can be used as the organic solvent. An about 30% to about 70% organic solvent may be used as the mixed solvent of water and the organic solvent. Extracting using an organic solvent may be performed at room temperature. The pharmaceutical composition may be acquired by concentrating the extract under reduced pressure at a low temperature. In addition, the pharmaceutical composition in the form of powder may be acquired by drying the extract.

Further, the present invention provides a pharmaceutical composition comprising pharmaceutically effective components having a molecular weight of not greater than 10,000 obtained from an extract of Cibotii Rhizoma, Ledebouriellae Radix, Achyranthes bidentatae Radix, Acanthopanacis Cortex, Eucommiae Cortex, and Glycine Semen nigra by allowing the extract to pass through an ultrafiltration(UF) membrane. The pharmaceutically effective components having the optimum concentrations for formulation can be acquired by removing impurities through UF. The pharmaceutical composition may be formulated in a tablet or capsule that is preferably administered that is administered in the range of from 30 to 1500 mg, more preferably from 30 to 600 mg, per dosage.

The present invention also provides a method of preparing a pharmaceutical composition comprising extracting Cibotii Rhizoma, Ledebouriellae Radix, Achyranthes bidentatae Radix, Acanthopanacis Cortex, Eucommiae Cortex, and Glycine Semen nigra using hot water, an organic solvent, or a mixed solvent thereof, and concentrating the extract under reduced pressure at a low temperature. In addition, the preparation method of the pharmaceutical composition according to the present invention mayfurther comprise, after the extracting, separating the extract by subjecting the same to passing through an ultrafiltration membrane to separate pharmaceutically effective components having a molecular weight of not greater than 10,000.

The preparation method of the pharmaceutical composition according to the present invention may further comprise, after the concentrating, formulating the extract in the form of a tablet or a capsule that is administered in the range of from 30 to 1500 mg per dosage.

### ADVANTAGEOUS EFFECTS

Since the pharmaceutical composition according to the present invention is extracted from naturally occurring raw medicinal substance, it is pharmacologically stable. The pharmaceutical composition according to the present invention has a potent therapeutic efficacy on arthritis, spinitis and osteoblastic cells. In addition, compared with the conventional NSAIDs, which had reportedly severe adverse side effects when they were administered for a long period, the pharmaceutical composition according to the present invention has substantially the same anti-inflammatory and analgesic effects, without causing potential side effects.

### BRIEF DESCRIPTION OF THE DRAWINGS

FIGS. 1a through 1d depict the anti-inflammatory effect of a pharmaceutical composition according to the present invention administered to mice with acute inflammation, in which FIG. 1a is a graphical representation in a case of using hot water as an extracting solvent (Example 1), FIG. 1b is a graphical representation in a case of using 30% ethanol as an extracting solvent (Example 2), FIG. 1c is a graphical representation in a case of using an extract having a molecular weight of not less than 10,000 obtained after filtration in hot water and UF (Example 3), and FIG. 1d is a graphical representation in a case of using an extract having a molecular weight of not greater than 10,000 obtained after filtration in hot water and UF (Example 4) (* and ** indicate P<0.05 and P<0.01 for control groups, respectively.);

FIGS. 2a through 2d depict the anti-inflammatory effect of a pharmaceutical composition according to the present invention administered to mice with acute inflammation for 2 weeks, in which FIG. 2a is a graphical representation in a case of using hot water as an extracting solvent (Example 1), FIG. 2b is a graphical representation in a case of using 30% ethanol as an extracting solvent (Example 2), FIG. 2c is a graphical representation in a case of using an extract having a molecular weight of not less than 10,000 obtained after filtration in hot water and UF (Example 3), and FIG. 2d is a graphical representation in a case of using an extract having a molecular weight of not greater than 10,000 obtained after filtration in hot water and UF (Example 4) (* and ** indicate P<0.05 and P<0.01 for control groups, respectively.);

FIGS. 3a through 3d depict the analgesic effect of a pharmaceutical composition according to the present invention administered to mice with pain, in which FIG. 3a is a graphical representation in a case of using hot water as an extracting solvent (Example 1), FIG. 3b is a graphical representation in a case of using 30% ethanol as an extracting solvent (Example 2), FIG. 3c is a graphical representation in a case of using an extract having a molecular weight of not less than 10,000 obtained after filtration in hot water and UF (Example 3), and FIG. 3d is a graphical representation in a case of using an extract having a molecular weight of not greater than 10,000 obtained after filtration in hot water and UF (Example 4) (* and ** indicate P<0.05 and P<0.01 for control groups, respectively.);

FIGS. 4a through 4d depict the anti-inflammatory effect of a pharmaceutical composition having a molecular weight of not greater than 10,000 obtained through UF, according to the present invention (Example 4), the pharmaceutical composition administered to mice with acute inflammation, in which FIG. 4a is a graphical representation in a case where the pharmaceutical composition is administered to the mouse in a dose of 30 mg/kg of body weight (Example 1), FIG. 4b is a graphical representation in a case where the pharmaceutical composition is administered to the mouse in a dose of 100 mg/kg of body weight (Example 2), FIG. 4c is a graphical representation in a case where the pharmaceutical composition is administered to the mouse in a dose of 300 mg/kg of body weight (Example 3), and FIG. 4d is a graphical representation in a case where the pharmaceutical composition is administered to the mouse in a dose of 600 mg/kg of body weight (Example 4) (* and ** indicate P<0.05 and P<0.01 for control groups, respectively.);

FIGS. 5a through 5d depict the anti-inflammatory effect of a pharmaceutical composition having a molecular weight of not greater than 10,000 obtained through UF, according to the present invention (Example 4), the pharmaceutical composition administered to mice with acute inflammation for 2 weeks, in which FIG. 5a is a graphical representation in a case where the pharmaceutical composition is administered to the mouse in a dose of 30 mg/kg of body weight (Example 1), FIG. 5b is a graphical representation in a case where the pharmaceutical composition is administered to the mouse in a dose of 100 mg/kg of body weight (Example 2), FIG. 5c is a graphical representation in a case where the pharmaceutical composition is administered to the mouse in a dose of 300 mg/kg of body weight (Example 3), and FIG. 5d is a graphical representation in a case where the pharmaceutical composition is administered to the mouse in a dose of 600 mg/kg of body weight (Example 4) (* and ** indicate P<0.05 and P<0.01 for control groups, respectively.);

FIG. 6 depicts the efficacy of a pharmaceutical composition on vascular permeability, the pharmaceutical composition having a molecular weight of not greater than 10,000 obtained through UF, according to the present invention (Example 4) (* indicates P<0.05 for control groups.);

FIG. 7 depicts the anti-inflammatory effect of a pharmaceutical composition having a molecular weight of not greater than 10,000 obtained through UF, according to the present invention (Example 4), the pharmaceutical composition administered to mice with chronic inflammation (** indicates P<0.01 for control groups.);

FIG. 8 depicts the analgesic effect of a pharmaceutical composition on the peripheral nerve system, the pharmaceutical composition having a molecular weight of not greater than 10,000 obtained through UF, according to the present invention (Example 4) (* and ** indicate P<0.05 and P<0.01 for control groups, respectively.);

FIG. 9 depicts the analgesic effect of a pharmaceutical composition on the central nerve system, the pharmaceutical composition having a molecular weight of not greater than 10,000 obtained through UF, according to the present invention (Example 4) (* and ** indicate P<0.05 and P<0.01 for control groups, respectively.);

FIG. 10 depicts the analgesic sensitivity of a pharmaceutical composition on the peripheral nerve system, the pharmaceutical composition having a molecular weight of not greater than 10,000 obtained through UF, according to the present invention (Example 4) (** indicates P<0.01 for control groups.);

FIGS. 11a through 11d depict the therapeutic effect of a pharmaceutical composition having a molecular weight of not greater than 10,000 obtained through UF, according to the present invention (Example 4), the pharmaceutical composition administered to mice with rheumatoid arthritis, in which FIG. 11a is a graphical representation in a case where the pharmaceutical composition is administered to the mouse in a dose of 30 mg/kg of body weight (Example 1), FIG. 11b is a graphical representation in a case where the pharmaceutical composition is administered to the mouse in a dose of 100 mg/kg of body weight (Example 2), FIG. 11c is a graphical representation in a case where the pharmaceutical composition is administered to the mouse in a dose of 300 mg/kg of body weight (Example 3), and FIG. 11d is a graphical representation in a case where the pharmaceutical composition is administered to the mouse in a dose of 600 mg/kg of body weight (Example 4) (* and ** indicate P<0.05 and P<0.01 for control groups, respectively.); and

FIGS. 12a and 12b depict viability and ALP activity showing the osteoblastic cell proliferating and activating effects of a pharmaceutical composition having a molecular weight of not greater than 10,000 obtained through UF, according to the present invention (Example 4) (* and ** indicate P<0.05 and P<0.01 for control groups, respectively.).

### BEST MODE FOR CARRYING OUT THE INVENTION

The present invention will be more specifically illustrated by the following examples and experiments. However, it should be understood that these examples and experiments do not intend to limit the scope of the present invention in any manner.

### Examples

### Example 1

### Preparation of Hot Water-Extract from Cibotii Rhizoma, Ledebouriellae Radix, Achyranthes Bidentatae Radix, Acanthopanacis Cortex, Eucommiae Cortex, and Glycine Semen nigra

4.167 g of Cibotii Rhizoma, 6.250 g of Ledebouriellae Radix, 6.250 g of Achyranthes bidentatae Radix, 6.250 g of Acanthopanacis Cortex, 2.083 g of Eucommiae Cortex and 4.167 g of Glycine Semen nigra were precisely weighed, followed by grinding the resultant raw substance mixture for one minute using a home-use crusher to make powder of the raw substance mixture. To the resultant product was added 50-fold distilled water based on the total weight of the raw substance mixture, and refluxed in hot water of approximately 100° C. for 3 hours and cooled for extraction. The cooled extract was filtered using a gauze and the resultant filtrate was concentrated in a water bath of approximately 45° C. under reduced pressure to prepare the title extract of the pharmaceutical composition according to the present invention.

### Example 2

### Preparation of 30% Ethanol-Extract from Cibotii Rhizoma, Ledebouriellae Radix, Achyranthes Bidentatae Radix, Acanthopanacis Cortex, Eucommiae Cortex, and Glycine Semen Nigra

4.167 g of Cibotii Rhizoma, 6.250 g of Ledebouriellae Radix, 6.250 g of Achyranthes bidentatae Radix, 6.250 g of Acanthopanacis Cortex, 2.083 g of Eucommiae Cortex and 4.167 g of Glycine Semen nigra were precisely weighed, followed by grinding the resultant raw substance mixture for one minute using a home crusher to make powder of the raw substance mixture. To the resultant product was added 30% ethanol (containing ethanol of 30% an extracting solvent) for leaching at room temperature for 3 hours. The extraction at room temperature for 3 hours was repeated twice, followed by filtrating using a gauze and concentrating in a water bath of approximately 45° C. under reduced pressure to prepare the title extract of the pharmaceutical composition according to the present invention.

### Example 3

### Isolation of Extract Having Molecular Weight of not Less than 10,000 Through UF (Ultra-Filtration)

2.778 g of Cibotii Rhizoma, 4.444 g of Ledebouriellae Radix, 4.444 g of Achyranthes bidentatae Radix, 4.444 g of Acanthopanacis Cortex, 1.389 g of Eucommiae Cortex and 2.778 g of Glycine Semen nigra were precisely weighed, followed by grinding the resultant raw substance mixture for one minute using a home crusher to make powder of the raw substance mixture. To the resultant product was added 50-fold distilled water based on the total weight of the raw substance mixture, and refluxed in hot water of approximately 100° C. for 3 hours and cooled for extraction. The resultant product was filtered using Whatman No. 2 filter paper and the obtained filtrate was further filtered using a 0.65 □ capsule-type filter. The filtrate was subjected to UF (100,000, TFF membrane) to obtain a filtrate having a molecular weight of not less than 100,000. Then, the resultant filtrate was again subjected to UF (10,000, TFF membrane) to obtain a filtrate having a molecular weight of not less than 10,000. The obtained filtrates were concentrated in a water bath of approximately 45° C. under reduced pressure to prepare the title extract of the pharmaceutical composition according to the present invention having a molecular weight of not less than 10,000.

### Example 4

### Isolation of Extract Having Molecular Weight of not Greater than 10,000 through Ultra-Filtration (UF)

The title extract of the pharmaceutical composition according to the present invention having a molecular weight of not greater than 10,000 was prepared in the same manner as in Example 3, except that a filtrate having a molecular weight of not greater than 10,000 was concentrated under reduced pressure.

### Experimental Materials and Methods

### A. Animals and Breeding Conditions

Male SD (Sprague-Dawley) albino rats weighing 140 to 200 g and ICR mice (Jeil Corp., Korea) weighing 20 to 25 g were used as experimental animals. The experimental animals were placed in animal breeding rooms with controlled environments of a temperature being 23±1° C., relative humidity being 55±15% and light intensity being 300 to 500 Lux with a 12:12 hour light-dark cycle (Neuropharmachology Research, Sungkyunkwan University, Korea) and allowed to acclimatize for over one week before experimental use. Animals had free access to water and solid food for feeding animals used in experiments (Samyang Corp., Korea). Visual symptoms demonstrated on the animals were observed and normal animals only were used in experiments.

### B. Preparation and Administration of Experimental Materials

The pharmaceutical compositions prepared in Examples were dissolved in saline (1 ml per 100 g of body weight) in each of doses of 100 and 300 mg/kg of body weight and forcibly administered using a needle (sonde) for oral administration.

Administration volumes were calculated based on body weights measured on the administration day and only saline (1 ml per 100 g of body weight) was administered as a control.

As a positive control drug, phenylbutazone suspended in a 0.5% methylcellulose solution was used in a dose of 50 mg/kg/5 ml for oral administration.

### C. Statistical Analysis

Verification of statistical significance for each test group was carried out in the following manner. Levene's test was carried out on data obtained for each test group to investigate variance homogeneity. When the variance homogeneity was verified, one way analysis of variance (ANOVA) was performed. When significant at p=0.05, Dunnett's t-test was performed for pairwise comparisons between treated and control groups (Procedure 1). In the absence of variance homogeneity, data transformation was performed. Then, Levene's test was carried out on the transformed data. When the variance homogeneity was verified, the same procedure as the procedure 1 is conducted for completion of verification of statistical significance. However, if the variance is inhomogeneous, a non-parametric analysis of variance (ANOVA) test was performed. If the result is significant, the statistical significance was tested using a Wilcoxon-Mann-Whitney rank sum test (Procedure 2).

### Experimental Example 1

### Acute Inflammation Test by Carrageenan-Induced Hind Paw Edema—Tested 30 Minutes after Injection

SD male albino rats weighing 150 to 170 g, which were fasted for 16 hours before experimental use, were used as test animals to induce edema by subcutaneously injecting 0.1 ml of a 1% type IV lambda carrageenan-saline solution as a platelet activator to the subplantar of the right hind paw of each of the albino rats (Winter et al., 1962). The paw volume was measured with a plethysmometer (Ugo Basile, Italy) 0.5, 1, 2, 4 and 6 hours after injection of carrageenan and compared with the paw volume measured before injection of carrageenan for calculation of an increase (%) of paw volume. In addition, an increase (%) of paw volume for a control group was compared with increases (%) of paw volume for treatment groups for obtaining a reduction ratio (%) of paw volume. The pharmaceutical compositions prepared in Examples 1 through 4 were orally administered to the control group 30 minutes before injection of carrageenan. The extent of paw edema (or swelling) was obtained based on the edema enhancement rate and edema inhibition rate measured using the following relationship. Table 1 shows measurement results of increased percent of paw volume (%). Phenylbutazone was used as a positive control.

TABLE 1

| | Dose | Increased percent of paw volume (%) | | | | |
|---|---|---|---|---|---|---|
| Group | (mg/kg) | 0.5 | 1 | 2 | 4 | 6 (h) |
| Control | 0 | 22.2 ± 3.9 | 30.0 ± 5.3 | 41.9 ± 6.1 | 61.7 ± 5.7 | 53.1 ± 3.0 |
| Example 1 | 100 | 17.4 ± 3.2 | 21.5 ± 3.2 | 28.7 ± 3.3 | 54.4 ± 6.3 | 50.3 ± 2.8 |
| | | 21.7 | 28.4 | 31.5 | 11.8 | 5.3 |
| | 300 | 10.0 ± 2.1 | 15.8 ± 2.6 | 22.4 ± 4.7 ** | 54.2 ± 6.1 | 45.4 ± 3.5 |
| | | 54.8 | 47.3 | 46.5 | 12.2 | 14.5 |
| Example 2 | 100 | 15.7 ± 1.6 | 17.8 ± 1.2 | 29.1 ± 2.0 | 56.0 ± 5.5 | 52.8 ± 1.7 |
| | | 29.4 | 40.6 | 30.4 | 9.2 | 0.7 |
| | 300 | 15.6 ± 3.3 | 20.6 ± 3.3 | 25.2 ± 2.4 * | 51.2 ± 6.1 | 47.7 ± 3.4 |
| | | 29.8 | 31.5 | 39.8 | 17.1 | 10.2 |
| Example 3 | 100 | 11.6 ± 1.5 | 14.8 ± 2.3 | 20.7 ± 2.0 ** | 50.0 ± 5.2 | 44.1 ± 3.9 |
| | | 47.6 | 50.8 | 50.6 | 18.9 | 17.0 |
| | 300 | 11.7 ± 1.6 | 14.3 ± 1.2 ** | 22.4 ± 2.1 ** | 54.3 ± 6.1 | 49.3 ± 4.3 |
| | | 47.3 | 52.3 | 46.6 | 12.0 | 7.2 |
| Example 4 | 100 | 16.0 ± 2.1 | 20.9 ± 1.4 | 31.5 ± 2.6 | 62.6 ± 1.7 | 54.6 ± 3.9 |
| | | 28.0 | 30.3 | 24.8 | −1.4 | −2.7 |
| | 300 | 7.3 ± 1.2 ** | 13.0 ± 2.2 * | 20.4 ± 1.8 ** | 52.9 ± 4.8 | 42.5 ± 4.6 |
| | | 66.9 | 56.8 | 51.3 | 14.3 | 20.1 |
| Phenyl- | 50 | 11.1 ± 0.8 * | 14.0 ± 0.9 ** | 25.0 ± 3.7 ** | 37.8 ± 4.3 | 35.0 ± 2.4 ** |
| butazone | | 49.9 | 53.2 | 40.3 | 38.8 | 34.2 |

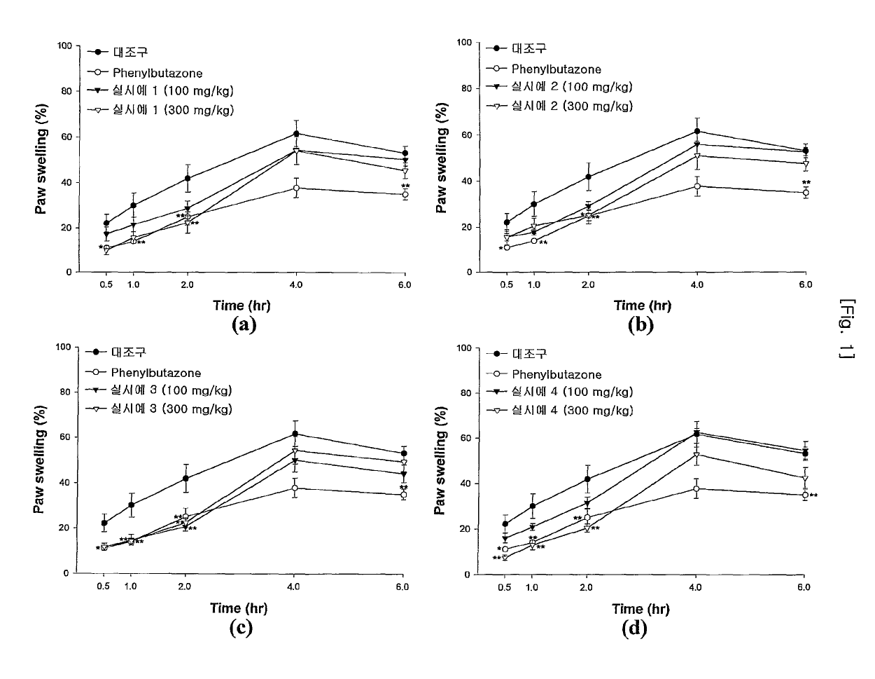

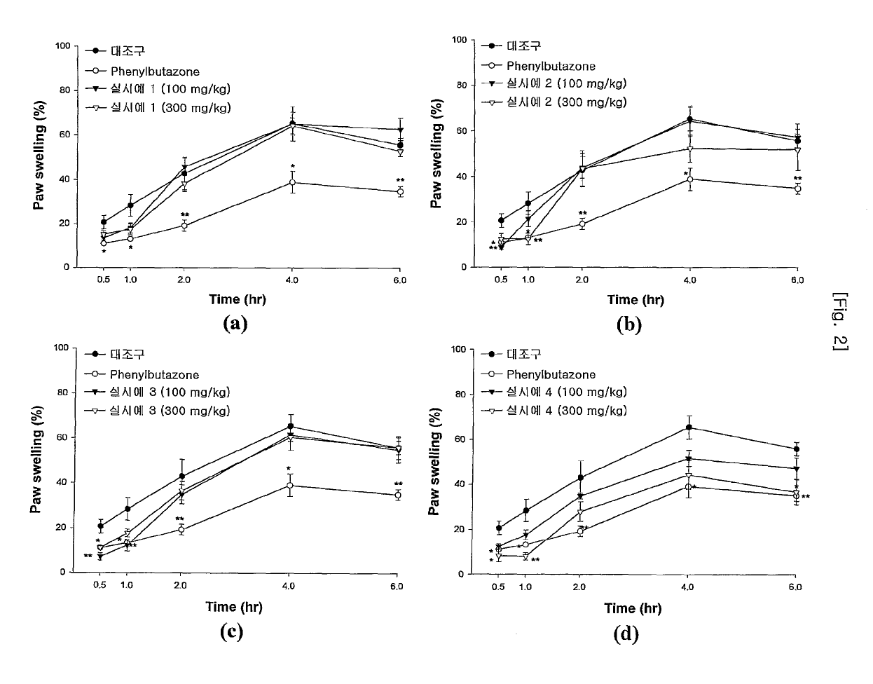

FIGS. 1a through 1d depict the anti-inflammatory effect of a pharmaceutical composition according to the present invention administered to mice with acute inflammation, as indicated by increased percent of paw swelling (%), in which FIG. 1a is a graphical representation in a case of using hot water as an extracting solvent (Example 1), FIG. 1b is a graphical representation in a case of using 30% ethanol as an extracting solvent (Example 2), FIG. 1c is a graphical representation in a case of using an extract having a molecular weight of not less than 10,000 obtained after filtration in hot water and UF (Example 3), and FIG. 1d is a graphical representation in a case of using an extract having a molecular weight of not greater than 10,000 obtained after filtration in hot water and UF (Example 4). Saline was used as a control and 50 mg/kg of phenylbutazone was used as a positive control.

As shown in Table 1 and FIG. 1, it could be noted that the control group to which only saline was administered as a preparation solvent showed 30.0% of an increase in the paw volume 1 hour after administration of carrageenan, 41.9% 2 hours after administration of carrageenan, and 61.7% 4 hours after administration of carrageenan, whereas the treatment groups of Examples 1 through 4 showed a significantly inhibitory effect. In comparison with a case of administering phenylbutazone, the treatment groups showed substantially the same inhibitory effects within a range of 2 hours.

### Experimental Example 2

### Acute Inflammation Test by Carrageenan-Induced Hind Paw Edema—Tested 2 Weeks after Injection

The pharmaceutical compositions prepared in Examples 1 through 4 were orally administered once at the same specified time point of the day for a period of 2 weeks, with the proviso that the experiment was carried out in the same manner as in Experimental Example 1. Table 2 shows measurement results of increased percent of paw volume (%).

TABLE 2

| | Dose | Increased percent of paw volume (%) | | | | |
|---|---|---|---|---|---|---|
| Group | (mg/kg) | 0.5 | 1 | 2 | 4 | 6 (h) |
| Control | 0 | 20.7 ± 3.0 | 28.4 ± 5.0 | 43.0 ± 7.4 | 65.4 ± 5.2 | 55.8 ± 3.0 |
| Example 1 | 100 | 13.7 ± 1.2 | 18.1 ± 2.4 | 46.0 ± 3.8 | 65.2 ± 7.8 | 62.8 ± 5.0 |
| | | (34.0) | (36.2) | (−27.1) | (−6.2) | (−16.5) |
| | 300 | 15.3 ± 1.6 | 17.5 ± 2.5 | 38.4 ± 3.6 | 64.4 ± 3.6 | 53.1 ± 2.4 |
| | | (26.2) | (38.4) | (−1.4) | (−9.4) | (−4.6) |
| Example 2 | 100 | 8.8 ± 0.9 ** | 21.4 ± 3.5 | 44.1 ± 4.8 | 64.5 ± 6.6 | 57.4 ± 5.8 |
| | | (57.2) | (24.5) | (−22.4) | (−10.3) | (−6.4) |
| | 300 | 12.6 ± 2.3 | 12.6 ± 2.5 ** | 43.7 ± 7.8 | 52.5 ± 5.9 | 52.0 ± 9.0 |
| | | (39.1) | (55.7) | (−15.4) | (2.4) | (3.6) |
| Example 3 | 100 | 7.1 ± 1.6 ** | 12.1 ± 2.6 ** | 34.8 ± 4.3 | 61.7 ± 3.0 | 54.8 ± 5.7 |
| | | (65.5) | (57.5) | (8.1) | (−0.3) | (−1.7) |
| | 300 | 11.1 ± 0.9 | 17.6 ± 1.7 | 36.6 ± 4.2 | 60.5 ± 5.9 | 55.8 ± 5.3 |
| | | (46.6) | (38.1) | (−4.3) | (−4.0) | (−10.9) |
| Example 4 | 100 | 12.4 ± 1.2 | 17.6 ± 2.2 | 34.8 ± 1.3 | 51.6 ± 3.5 | 47.0 ± 4.7 |
| | | (40.2) | (37.9) | (8.1) | (16.1) | (12.8) |
| | 300 | 8.2 ± 2.7 * | 8.0 ± 1.7 ** | 27.9 ± 4.3 | 44.2 ± 6.4 | 36.5 ± 5.5 * |
| | | (60.5) | (72.0) | (26.4) | (28.1) | (32.3) |
| Phenyl- | 50 | 11.0 ± 0.5 * | 13.2 ± 0.8 * | 19.3 ± 2.4 ** | 39.1 ± 5.0 * | 35.0 ± 2.4 ** |
| Butazone | | (46.6) | (53.5) | (49.2) | (36.4) | (35.2) |

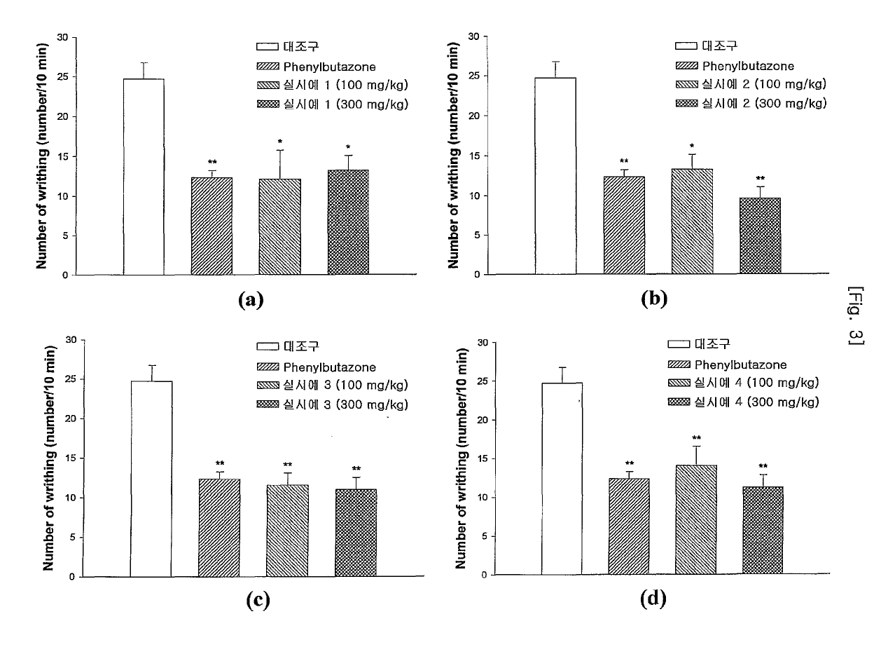

FIGS. 2a through 2d depict the anti-inflammatory effect of a pharmaceutical composition according to the present invention administered to mice with acute inflammation for 2 weeks, as indicated by increased percent of paw swelling (%), in which FIG. 2a is a graphical representation in a case of using hot water as an extracting solvent (Example 1), FIG. 2b is a graphical representation in a case of using 30% ethanol as an extracting solvent (Example 2), FIG. 2c is a graphical representation in a case of using an extract having a molecular weight of not less than 10,000 obtained after filtration in hot water and UF (Example 3), and FIG. 2d is a graphical representation in a case of using an extract having a molecular weight of not greater than 10,000 obtained after filtration in hot water and UF (Example 4) (* and ** indicate P<0.05 and P<0.01 for controls, respectively.). Saline was used as a control and 50 mg/kg of phenylbutazone was used as a positive control.

### Experimental Example 3

### Analgesic Effect Test

The pharmaceutical compositions prepared in Examples 1 through 4 were orally administered to ICR (Institute of Cancer Research) male mice weighing 25 to 28 g, which were fasted for 12 hours before experimental use. After 30 minutes of administration, 0.7% acetic acid-saline solution was intraperitoneally injected to the mice in a dose of 0.1 mg per 10 g of body weight and from 5 minutes after administration, the number of writhings occurring to each mouse was counted for 10 minutes (Koster et al., 1959) and the results are summarized in Table 3. Phenylbutazone was used as a positive control.

TABLE 3

| | Dose | | |
|---|---|---|---|
| Group | (mg/kg) | Number of writhing | Inhibition rate (%) |
| Control | 0 | 24.7 ± 2.0 | |
| Example 1 | 100 | 12.1 ± 3.6 * | 50.9 |
| | 300 | 13.3 ± 1.8 * | 46.4 |
| Example 2 | 100 | 13.3 ± 1.8 * | 46.2 |
| | 300 | 9.6 ± 1.4 ** | 61.0 |
| Example 3 | 100 | 11.6 ± 1.5 ** | 53.2 |
| | 300 | 11.0 ± 1.5 ** | 55.5 |
| Example 4 | 100 | 14.0 ± 2.5 ** | 43.3 |
| | 300 | 11.2 ± 1.6 ** | 54.6 |
| Phenylbutazone | 50 | 12.3 ± 0.9 ** | 50.1 |

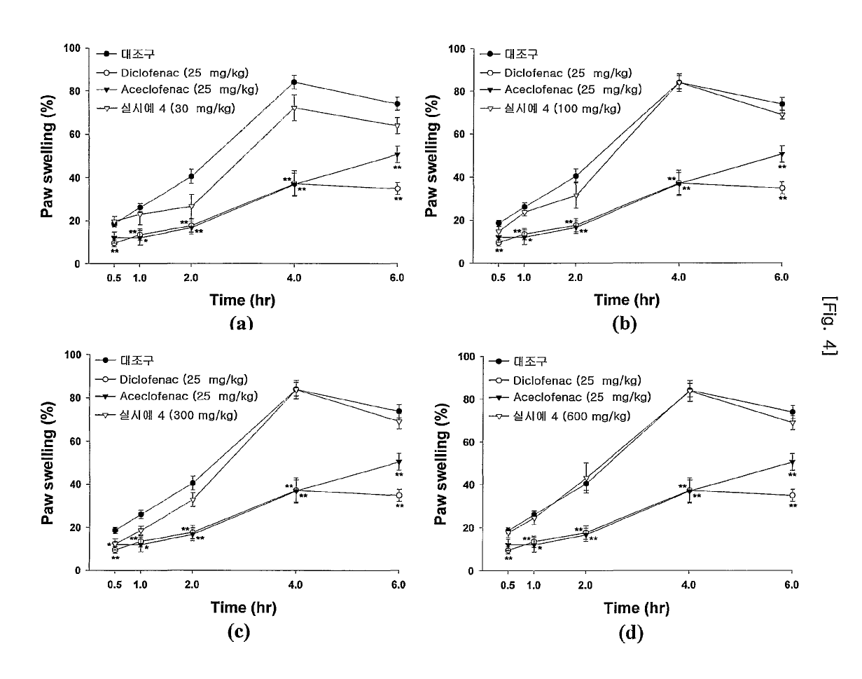

FIGS. 3a through 3d depict the analgesic effect of a pharmaceutical composition according to the present invention administered to mice with pain, in which FIG. 3a is a graphical representation in a case of using hot water as an extracting solvent (Example 1), FIG. 3b is a graphical representation in a case of using 30% ethanol as an extracting solvent (Example 2), FIG. 3c is a graphical representation in a case of using an extract having a molecular weight of not less than 10,000 obtained after filtration in hot water and UF (Example 3), and FIG. 3d is a graphical representation in a case of using an extract having a molecular weight of not greater than 10,000 obtained after filtration in hot water and UF (Example 4). Saline was used as a control and 50 mg/kg of phenylbutazone was used as a positive control.

As shown in FIG. 3, the pharmaceutical composition according to the present invention exhibited substantially the same analgesic effect as phenylbutazone, which has potent analgesic effect.

### Experimental Example 4

### Acute Inflammation Test by Carrageenan-Induced Hind Paw Edema—Tested 30 Minutes after Injection

In order to examine the inhibitory effect of the pharmaceutical composition prepared in Example 4 of the present invention on acute inflammation induced by carrageenan, the pharmaceutical composition was administered in doses of 30, 100, 300, 600 mg/kg in the same manner as in Experimental Example 1 and the results are summarized in Table 4.

TABLE 4

| | Dose | Increased percent of paw volume (%) | | | | |
|---|---|---|---|---|---|---|
| Group | (mg/kg) | 0.5 | 1 | 2 | 4 | 6 (h) |
| Control | 0 | 18.63 ± 1.4 | 26.1 ± 1.9 | 40.5 ± 3.2 | 84.1 ± 3.2 | 73.9 ± 3.0 |
| Example 4 | 30 | 19.8 ± 2.2 | 23.1 ± 4.9 | 26.6 ± 5.6 | 72.2 ± 6.0 | 63.84 ± 3.7 |
| | | −8.8 | 11.6 | 34.5 | 14.1 | 13.6 |
| | 100 | 14.8 ± 2.4 | 23.6 ± 1.7 | 31.62 ± 6.1 | 84.0 ± 4.3 | 68.9 ± 2.0 |
| | | 18.8 | 9.5 | 22 | 0.1 | 6.8 |
| | 300 | 12.4 ± 1.2 * | 18.6 ± 2.0 | 32.8 ± 3.2 | 83.9 ± 4.2 | 69.3 ± 3.5 |
| | | 33.5 | 28.8 | 19.1 | 0.2 | 6.3 |
| | 600 | 17.7 ± 2.3 | 24.6 ± 3.1 | 43.2 ± 7.1 | 83.7 ± 4.8 | 69.0 ± 3.3 |
| | | 2.6 | 5.9 | −6.5 | 0.4 | 6.6 |
| Diclofenac | 25 | 9.6 ± 1.6 ** | 13.6 ± 2.6 ** | 17.7 ± 3.1 ** | 37.24 ± 5.9 ** | 34.89 ± 2.8 ** |
| | | 47.5 | 48.0 | 56.3 | 55.7 | 52.8 |
| Aceclofenac | 25 | 12.1 ± 2.6 | 12.1 ± 3.5 * | 16.7 ± 3.0 ** | 36.9 ± 5.1 ** | 50.6 ± 3.9 ** |
| | | 34.8 | 53.7 | 58.7 | 56.1 | 31.5 |

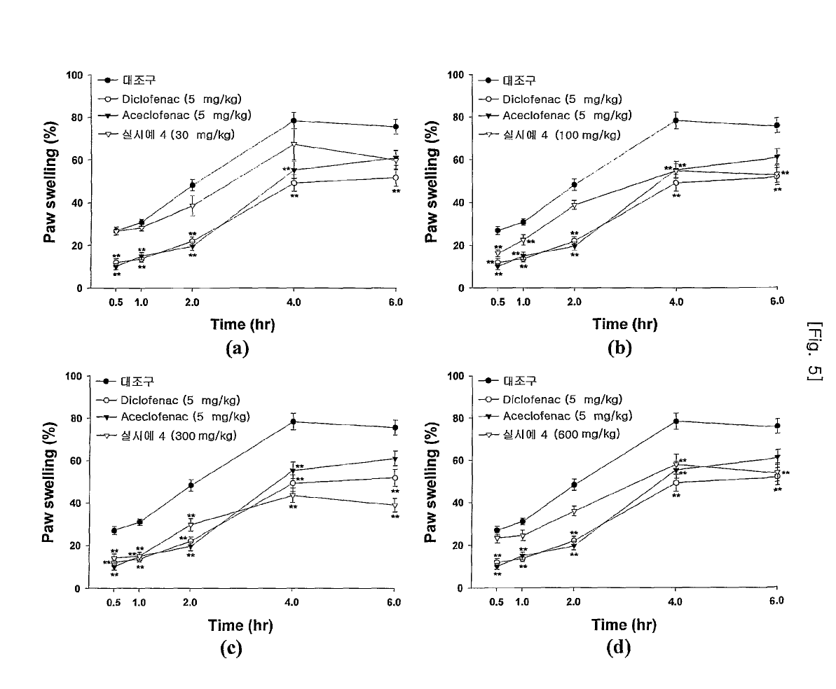

FIGS. 4a through 4d depict the anti-inflammatory effect of a pharmaceutical-composition having a molecular weight of not greater than 10,000 obtained through UF, according to the present invention (Example 4), the pharmaceutical composition administered to mice with acute inflammation, in which FIG. 4a is a graphical representation in a case where the pharmaceutical composition is administered to the mouse in a dose of 30 mg/kg of body weight (Example 1), FIG. 4b is a graphical representation in a case where the pharmaceutical composition is administered to the mouse in a dose of 100 mg/kg of body weight (Example 2), FIG. 4c is a graphical representation in a case where the pharmaceutical composition is administered to the mouse in a dose of 300 mg/kg of body weight (Example 3), and FIG. 4d is a graphical representation in a case where the pharmaceutical composition is administered to the mouse in a dose of 600 mg/kg of body weight (Example 4). Saline was used as a control and each 25 mg/kg of diclofenac and aceclofenac were used as positive controls. As shown in FIGS. 4a through 4d, the pharmaceutical composition according to the present invention exhibited a slight inhibitory effect.

### Experimental Example 5

### Acute Inflammation Test by Carrageenan-Induced Hind Paw Edema—Tested 2 Weeks after Injection

After the pharmaceutical composition according to the present invention prepared in Example 4 was administered to mice for 2 weeks, the experiment was carried out in the same manner as in Experimental Example 4. The results are summarized in Table 5.

TABLE 5

| | Dose | Increased percent of paw volume (%) | | | | |
|---|---|---|---|---|---|---|
| Group | (mg/kg) | 0.5 | 1 | 2 | 4 | 6 (h) |
| Control | 0 | 27.0 ± 1.9 | 31.0 ± 1.5 | 48.3 ± 2.7 | 78.3 ± 3.9 | 75.6 ± 3.5 |
| Example 4 | 30 | 26.6 ± 1.7 | 28.5 ± 1.5 | 38.7 ± 4.7 | 67.4 ± 7.3 | 59.9 ± 4.5 |
| | | 1.3 | 8.0 | 19.9 | 14.0 | 20.8 |
| | 100 | 16.3 ± 1.5 ** | 22.5 ± 2.4 * | 38.9 ± 2.0 | 54.8 ± 3.4 ** | 52.9 ± 4.1 ** |
| | | 39.6 | 27.4 | 19.4 | 30.0 | 30.0 |
| | 300 | 14.2 ± 2.0 ** | 15.2 ± 2.0 ** | 29.7 ± 3.0 ** | 43.6 ± 3.4 ** | 38.9 ± 3.2 ** |
| | | 47.5 | 50.9 | 38.5 | 44.4 | 48.6 |
| | 600 | 23.4 ± 2.4 | 24.5 ± 2.4 | 35.9 ± 2.3 | 58.0 ± 4.7 ** | 53.7 ± 4.4 ** |
| | | 13.3 | 21.0 | 25.7 | 26.0 | 28.9 |
| Diclofenac | 5 | 12.0 ± 1.8 ** | 13.7 ± 1.4 ** | 22.0 ± 2.0 ** | 49.2 ± 3.9 ** | 51.8 ± 4.0 ** |
| | | 55.5 | 55.6 | 54.4 | 37.1 | 31.50 |
| Aceclofenac | 5 | 10.1 ± 1.4 ** | 15.1 ± 1.7 ** | 19.7 ± 2.0 ** | 55.3 ± 3.9 ** | 61.0 ± 3.5 |
| | | 62.6 | 51.4 | 59.2 | 29.4 | 19.4 |

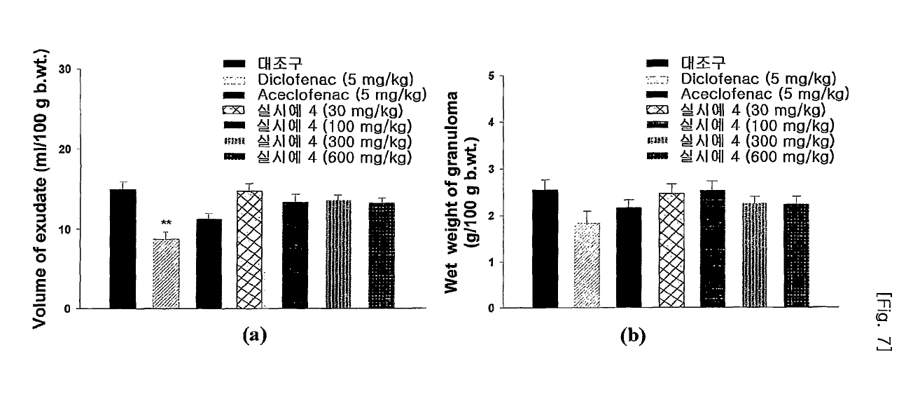

FIGS. 5a through 5d depict the anti-inflammatory effect of a pharmaceutical composition having a molecular weight of not greater than 10,000 obtained through UF, according to the present invention (Example 4), the pharmaceutical composition administered for 2 weeks to mice with acute inflammation, in which FIG. 5a is a graphical representation in a case where the pharmaceutical composition is administered to the mouse in a dose of 30 mg/kg of body weight (Example 1), FIG. 5b is a graphical representation in a case where the pharmaceutical composition is administered to the mouse in a dose of 100 mg/kg of body weight (Example 2), FIG. 5c is a graphical representation in a case where the pharmaceutical composition is administered to the mouse in a dose of 300 mg/kg of body weight (Example 3), and FIG. 5d is a graphical representation in a case where the pharmaceutical composition is administered to the mouse in a dose of 600 mg/kg of body weight (Example 4). Saline was used as a control and each 25 mg/kg of diclofenac and aceclofenac were used as positive controls.

As shown in FIG. 5, the pharmaceutical composition according to the present invention exhibits an anti-inflammatory effect in a dose range of from of 300 mg/kg or higher, which is substantially the same as or better than diclofenac and aceclofenac as positive controls.

### Experimental Example 6

### Acetic Acid-Induced Vascular Permeability Test

The pharmaceutical composition prepared in Example 4 was orally administered to male ICR mice weighing 25 to 28 g by the Whittle (1964) method, which were fasted for 12 hours before experimental use. 30 minutes after administration, 0.1 ml per 10 g of body weight of a 2.5% Evans blue solution was administered through the tail vein of each mouse and 20 minutes after administration, 0.1 ml per 10 g of body weight of a 0.6% acetic acid-saline solution was intraperitoneally injected. After a lapse of 20 minutes, mice were sacrificed by cervical dislocation, 5 ml of saline was infused into the abdominal cavity of each mouse to then carry out a lavage by softly shaking the abdomen, thereby obtaining an abdominal cavity lavage fluid. The lavage fluid was centrifuged at 2,000 rpm for 10 minutes, and the supernatants were retained for evaluation of Evans blue dye extravasation from circulation as assessed by spectrophotometry of the supernatant at 610 nm. Each 25 mg/kg of body weight of diclofenac and aceclofenac were used as positive control drugs.

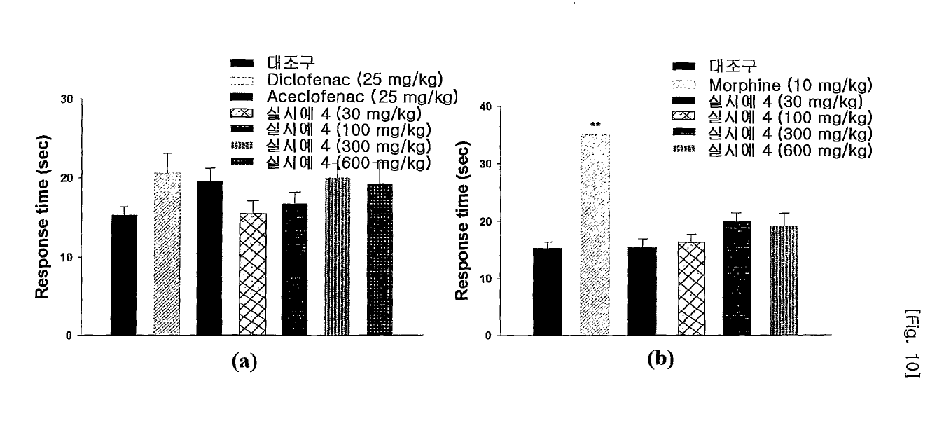

FIG. 6 depicts the inhibitory efficacy of a pharmaceutical composition on vascular permeability, as indicated by leakage of dye, the pharmaceutical composition having a molecular weight of not greater than 10,000 obtained through UF, according to the present invention.

As shown in FIG. 6, the pharmaceutical composition according to the present invention exhibits a vascular permeability inhibitory effect substantially the same as or better than diclofenac and aceclofenac as positive controls.

### Experimental Example 7

### Chronic Inflammation Test by Carrageenan-Induced Granuloma

8 ml of air was subcutaneously injected into the back of each of SD male albino rats weighing 180 to 200 g to make a hemi-spherical air pouch by a process proposed by Tsurufuji et al. (1979). After a lapse of 24 hours, 4 ml of a 2% carrageenan-saline solution was infused into the air pouch. In treatment groups and control group, the pouch was cut on Day 8 and the volume of exudate and the wet weight of granuloma were measured for comparison. The pharmaceutical composition prepared in Example 4 was administered once daily at the same specified time point of the day for 7 consecutive days (Day 1 through Day 7), and 5 mg/kg of body weight of diclofenac and 5 mg/kg of body weight of aceclofenac were used as positive controls.

FIGS. 7a and 7b depict the anti-inflammatory effect of a pharmaceutical composition having a molecular weight of not greater than 10,000 obtained through UF, according to the present invention.

As shown in FIGS. 7a and 7b, the pharmaceutical compositions prepared in Examples of the present invention have volumes of exudate and wet weight of granuloma higher than diclofenac and aceclofenac but lower than in saline as a control.

### Experimental Example 8

### Test of Analgesic Effect on peripheral Nerve System by Acetic Acid-Induced Writhing Syndrome

25 mg/kg of body weight of diclofenac and 25 mg/kg of body weight of aceclofenac were used as positive control drugs. The pharmaceutical composition prepared in Example 4 were intraperitoneally administered to ICR male mice weighing 25 to 28 g, which were fasted for 12 hours before experimental use. After 30 minutes of administration, 0.7% acetic acid-saline solution was intraperitoneally injected to the mice in a dose of 0.1 mg per 10 g of body weight and from 5 minutes after administration, the number of writhings occurring to each mouse was counted for 10 minutes (Koster et al., 1959) and the results are summarized in Table 3. Phenylbutazone was used as a positive control drug.

FIG. 8 depicts the analgesic effect of a pharmaceutical composition on the peripheral nerve system, the pharmaceutical composition having a molecular weight of not greater than 10,000 obtained through UF, according to the present invention.

As shown in FIG. 8, the pharmaceutical composition according to the present invention exhibits an analgesic effect substantially the same as or better than diclofenac and aceclofenac as positive controls.

### Experimental Example 9

### Test of Analgesic Effect on Central Nerve System using Hot Plate

Male ICR mice weighing 25 to 28 g, which were fasted for 12 hours before experimental use, were used as test animals. A pain reaction was carried in the following manner. The mice were carefully placed on a hot plate(Ugo Basile, Italy) at approximately 55° C. until they show a painful sensation by licking their paws or jumping and the response time (sec) for these sensations to occur is recorded (Woolfe and MacDonald, 1944). 30 minutes prior to the experiment, the pharmaceutical composition prepared in Example 4 was orally administered to the mice. 25 mg/kg of body weight of diclofenac, 25 mg/kg of body weight of aceclofenac, and 10 mg/kg of body weight of morphine were used as positive control drugs.

FIG. 9 depicts the analgesic effect of a pharmaceutical composition on the central nerve system, the pharmaceutical composition having a molecular weight of not greater than 10,000 obtained through UF, according to the present invention.

As shown in FIG. 9, the pharmaceutical composition according to the present invention exhibits an analgesic effect in a dose range from 300 mg/kg, which is substantially the same as or better than diclofenac and aceclofenac as positive control drugs.

### Experimental Example 10

### Test of Analgesic Effect on Peripheral Nerve System by Randall-Selitto Assay

The experiment was carried out according to a method proposed by Randall and Selitto (1957). A 20% Brewer's yeast suspension was subcutaneously injected to the subplantar of each of SD male albino rats weighing 220 to 240 g, which were fasted for 18 hours before experimental use, to induce edema. When a mechanical force of 16 g/sec was applied to the dorsum of the albino rat's paw for stimulation using an analgesymeter (Ugo Basile Co. Ltd., Italy), a nociceptive threshold was determined by multiplying a moving distance of a wedge-shaped piston with a weight of the piston at a time for the rat to withdraw its paw from the stimulation. 2 hours after administration of the yeast suspension, the pharmaceutical composition prepared in Example 4 was orally administered to the rats and nociceptive thresholds were measured at 0, 0.5, 1, 2, 3 and 4 hours after administration of the yeast suspension. Each 25 mg/kg of body weight of diclofenac and 25 mg/kg of body weight of aceclofenac were used as positive control drugs.

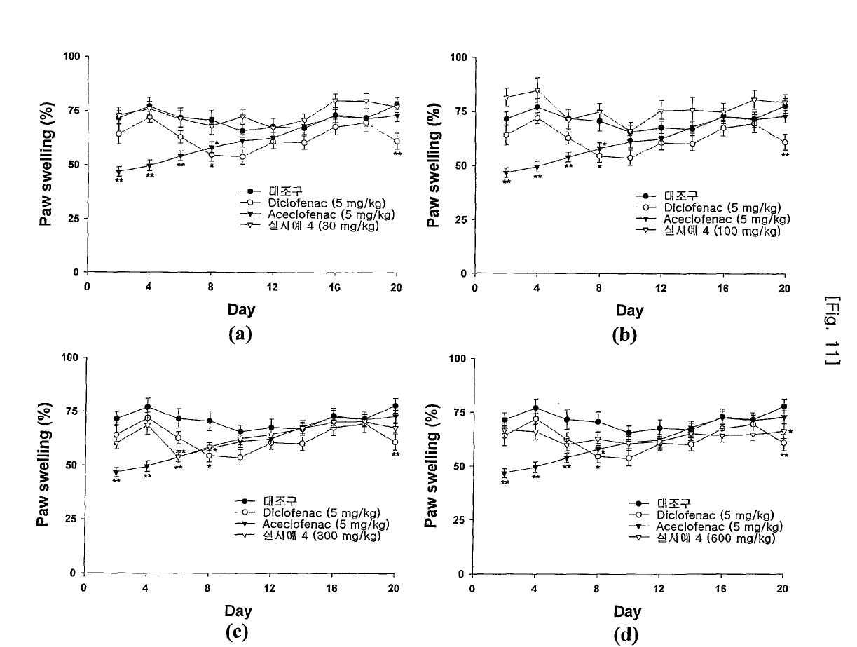

FIG. 10 depicts the pain sensitivity of a pharmaceutical composition on the peripheral nerve system, as indicated by a response time (sec), the pharmaceutical composition having a molecular weight of not greater than 10,000 obtained through UF, according to the present invention.

As shown in FIG. 10, the pharmaceutical composition according to the present invention exhibits a significant therapeutic effect in a dose range of from 300 mg/kg or higher.

### Experimental Example 11

### Adjuvant-Induced Rheumatoid Arthritis Test

0.1 ml of a complete Freund's adjuvant (CFA) containing mycobacterium tuberculosis in a concentration of 1 mg/ml was subcutaneously injected to the subplantar of the right hind paw of each of albino rats. The pharmaceutical composition prepared in Example 4 was orally administered to the rats for 20 consecutive days and edema rates were calculated once every other day using a plethysmometer.

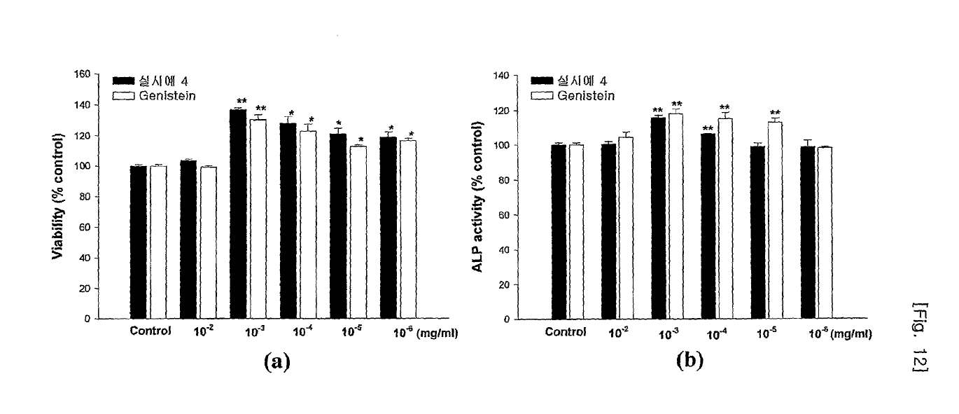

FIGS. 11a through 11d depict the therapeutic effect of a pharmaceutical composition having a molecular weight of not greater than 10,000 obtained through UF, according to the present invention (Example 4), as indicated by paw swelling (%), the pharmaceutical composition administered to mice with rheumatoid arthritis, in which FIG. 11a is a graphical representation in a case where the pharmaceutical composition is administered to the mouse in a dose of 30 mg/kg of body weight (Example 1), FIG. 11b is a graphical representation in a case where the pharmaceutical composition is administered to the mouse in a dose of 100 mg/kg of body weight (Example 2), FIG. 11c is a graphical representation in a case where the pharmaceutical composition is administered to the mouse in a dose of 300 mg/kg of body weight (Example 3), and FIG. 11d is a graphical representation in a case where the pharmaceutical composition is administered to the mouse in a dose of 600 mg/kg of body weight. Saline was used as a control and each 5 mg/kg of diclofenac and aceclofenac were used as positive controls.

As shown in FIG. 11, the pharmaceutical composition according to the present invention exhibits a rheumatoid arthritis therapeutic effect in a dose range of from 300 mg/kg or higher, which is substantially the same as or better than diclofenac and aceclofenac as positive controls.

### Experimental Example 12

### Osteoblastic Cell Proliferation and Activity Test Selection and Culture of Osteoblastic Cells

The MG-63 cell line, which has similar properties to osteoblastic cells, was distributed from Korean Cell Line Bank (KCLB) affiliated to the Cancer Research Institute, School of Medicine, Seoul National University for experimental use. The MG-63 cell line was incubated with a DMEM medium supplemented with 10% FBS (Fetal Bovine Serum), 100 unit/ml of penicillin, 100□/ml of streptomycin at 37° C. using a humidified 5% CO₂ incubator. The culture was fed with a fresh medium 2 to 3 times a week and sub-cultured once a week. Since cells tend to grow as monolayers in culture flasks, the monolayers were washed away using a 0.25% trypsin solution at a sub-culture time.

1) Proliferation of Osteoblastic Cells

After incubating the MG-63 cell line, the pharmaceutical composition prepared in Example 4 was dissolved in PBS, transferred to 10 wells with various concentrations ranging from 1×10⁻⁶ to 10⁻³ mg/ml and incubated for 48 hours. Following the incubation for 48 hours, 4 hour incubation was performed using a WelCount™ Cell Viability Assay Kit (JBI, Korea) under the same conditions as described above. The absorbance level measured at 650 was subtracted from the absorbance level measured at 450 nm to determine the viability of osteoblastic cells. The absorbance levels were measured using an ELISA reader. Genistein was used as a positive control.

FIG. 12a depicts the osteoblastic cell proliferating effect of a pharmaceutical composition having a molecular weight of not greater than 10,000 obtained through UF, according to the present invention.

2) Alkaline Phosphatase Activity of Osteoblastic Cell

24 hours after incubation of MG-63 cell line, the pharmaceutical composition prepared in Example 4 was dissolved in PBS, transferred to 10 wells with various concentrations ranging from 1×10⁻⁶ to 10⁻³ mg/ml and incubated for 48 hours. Following the 48 hour incubation, the incubated cells were washed 3 times with PBS and 500 □ of 0.1% triton X-100 was added to each well, followed by sonification for dissolving the cells. A yellow, alkaline p-nitrophenyl phosphate (pNPP) liquid substrate (Sigma Chemical Co.) was used to measure the ALP activity of the cell, and a protein content of the cell was measured using a Bio-Rad Protein assay kit. Genistein was used as a positive control.

FIG. 12b depicts the osteoblastic cell activating effect of a pharmaceutical composition having a molecular weight of not greater than 10,000 obtained through UF, according to the present invention.

3) Type IV Collagenase/Gelatinase Activity 24 hours after incubation of MG-63 cell line in a 12-well culture plate, the pharmaceutical composition prepared in Example 4 was dissolved in PBS and transferred to 5 wells with various concentrations ranging from 1×10⁻⁶ to 10⁻³ mg/ml. The culture fluid was fed with serum-free medium and further incubated for 72 hours. The gelatinase activity was evaluated by passing through a SDS-PAGE using a zymogram gel containing 0.1% gelatin. After electrophoresis, the gelatinase was renatured in a renaturing buffer for 30 minutes at room temperature, stabilized in a developing buffer for further 30 minutes, and incubated in a new developing buffer overnight at 37° C. The gel was stained in a solution of 0.5% Coomassie Brilliant Blue G-250 and destained with a destaining solution containing 30% MeOH and 10% acetic acid to visualize clear bands representing Type IV collagenase and gelatinase activities. The Type IV collagenase and gelatinase activities were visualized as clear bands against the dark blue background of the stained portions, as quantified by densitometric scanning of bands measured as densities thereat (Science Lab 98 Image Guage, version 3.12, Fuji Photo Film Co., Ltd., Tokyo, Japan).

As shown in FIG. 12, the pharmaceutical composition prepared in Example 4 exhibits substantially the same effect as Genistein^{R} as a control from the viewpoints of osteoblastic cell proliferation and ALP activity.

### INDUSTRIAL APPLICABILITY

Since the pharmaceutical composition according to the present invention is extracted from naturally occurring raw medicinal substance, it is pharmacologically stable. In addition, the pharmaceutical composition according to the present invention is effective in preventing or treating inflammation, pain, arthritis and spinitis, and proliferating or activating osteoblastic cells, unlike the conventional NSAIDs, which may reportedly produce unwanted side effects when long-term administration is involved.

What is claimed is:

1-12. (canceled)

13. A pharmaceutical compound containing pharmaceutically effective components comprising
extracted components from Cibotii Rhizoma, Ledebouriellae Radix, Achyranthes bidentatae Radix, Acanthopanacis Cortex, Eucommiae Cortex, and Glycine Semen nigra, wherein said pharmaceutically effective components being extracted from Cibotii Rhizoma, Ledebouriellae Radix, Achyranthes bidentatae Radix, Acanthopanacis Cortex, Eucommiae Cortex, and Glycine Semen nigra have ratio by weight of 1:0.5˜3:0.5˜3:0.5˜3:0.1˜2:0.5˜2.

14. The pharmaceutical compound claimed in claim 13, wherein said pharmaceutically effective components are extracted from Cibotii Rhizoma, Ledebouriellae Radix, Achyranthes bidentatae Radix, Acanthopanacis Cortex, Eucommiae Cortex, and Glycine Semen nigra using hot water, an organic solvent, or a mixed solvent thereof.

15. The pharmaceutical compound claimed in claim 14, wherein the pharmaceutically effective components are filtered by an ultra filtration membrane to have a molecular weight of not greater than 10,000.

16. The pharmaceutical compound claimed in claim 15, wherein said pharmaceutical composition is in the form of a tablet or a capsule administered in the range of from 30 to 1500 mg per dosage.

17. The pharmaceutical compound claimed in claim 13, wherein said pharmaceutical composition is an anti-inflammatory agent.

18. The pharmaceutical compound claimed in claim 13, wherein said pharmaceutical composition is an arthritis therapeutic agent.

19. The pharmaceutical compound claimed in claim 13, wherein said pharmaceutical composition is a spinitis therapeutic agent.

20. The pharmaceutical compound claimed in claim 13, wherein said pharmaceutical composition is an osteoblastic cell proliferating and activating agent.

21. A method of producing a pharmaceutical compound, the method comprising the steps of:
extracting Cibotii Rhizoma, Ledebouriellae Radix, Achyranthes bidentatae Radix, Acanthopanacis Cortex, Eucommiae Cortex, and Glycine Semen nigra using hot water, an organic solvent, or a mixed solvent thereof; and concentrating the extract under reduced pressure at a low temperature,
separating the extract by subjecting the same to passing through an ultrafiltration membrane to separate pharmaceutically effective components having a molecular weight of not greater than 10,000.
concentrating formulating the extract in the form of a tablet or a capsule that is administered in the range of from 30 to 1500 mg per dosage.
